(19) United States
(12) Patent Application Publication
(10) Pub. No.: US 2025/0217970 A1
(43) Pub. Date: Jul. 3, 2025

(54) RISK PREDICTION OF HEART FAILURE

(71) Applicants: Case Western Reserve University, Cleveland, OH (US); UH Cleveland Medical Center, Cleveland, OH (US)

(72) Inventors: Shuo Li, Cleveland, OH (US); Sadeer Al-Kindi, Lyndhurst, OH (US); Sanjay Rajagopalan, Shaker Heights, OH (US); David L. Wilson, Cleveland Heights, OH (US); Ammar Hoori, Westlake, OH (US)

(21) Appl. No.: 18/651,752

(22) Filed: May 1, 2024

Related U.S. Application Data
(60) Provisional application No. 63/615,310, filed on Dec. 28, 2023.

(51) Int. Cl. G06T 7/00; A61B 6/00; A61B 6/03; A61B 6/50; G16H 50/30

(52) U.S. Cl. CPC G06T 7/0012; A61B 6/032; A61B 6/503; A61B 6/5217; G16H 50/30; G06T 2207/10081; G06T 2207/20081; G06T 2207/20084; G06T 2207/30048

(57) ABSTRACT

The present disclosure, in some embodiments, relates to a method. The method includes accessing digitized imaging data stored in a memory. The digitized imaging data corresponds to a patient. A plurality of pathophysiological pathway related features are extracted from the digitized imaging data. The plurality of pathophysiological pathway related features correspond to one or more pathophysiological pathways relating to heart failure. The plurality of pathophysiological pathway related features are provided to a machine learning stage. The machine learning stage is configured to generate a medical prediction of heart failure risk for the patient using the plurality of pathophysiological pathway related features.

### CROSS-REFERENCE TO RELATED APPLICATIONS

This application claims the benefit of priority from U.S. Provisional Patent Application Ser. No. 63/615,310 filed on Dec. 28, 2023 and entitled RADIOMICS-BASED RISK PREDICTION OF HEART FAILURE, the disclosure of which is hereby incorporated by reference in its entirety.

### BACKGROUND

In recent years, there has been significant interest in developing machine vision tools for interrogating medical images. Machine vision tools are computer systems that utilize artificial intelligence to analyze medical images. Such tools have the potential to improve health care for patients.

### BRIEF DESCRIPTION OF THE DRAWINGS

The accompanying drawings, which are incorporated in and constitute a part of the specification, illustrate various example operations, apparatus, methods, and other example embodiments of various aspects discussed herein. It will be appreciated that the illustrated element boundaries (e.g., boxes, groups of boxes, or other shapes) in the figures represent one example of the boundaries. One of ordinary skill in the art will appreciate that, in some examples, one element can be designed as multiple elements or that multiple elements can be designed as one element. In some examples, an element shown as an internal component of another element may be implemented as an external component and vice versa. Furthermore, elements may not be drawn to scale.

FIG. 1 illustrates some embodiments of a heart failure assessment apparatus comprising a machine learning stage configured to utilize pathophysiological pathway related features to generate a medical prediction of heart failure risk.

FIG. 2 illustrates some additional embodiments of a heart failure assessment apparatus comprising a machine learning stage configured to utilize pathophysiological pathway related features to generate a medical prediction of heart failure risk.

FIG. 3 illustrates a table showing some exemplary pathophysiological pathway related features that may be utilized by a disclosed heart failure assessment apparatus to generate a medical prediction of heart failure risk.

FIG. 4 illustrates a flow diagram showing some embodiments of a method of utilizing pathophysiological pathway related features to generate a medical prediction of heart failure risk for a patient.

FIGS. 5A-5B illustrate figures of various embodiments corresponding to a disclosed automated segmentation of digitized images.

FIG. 6 illustrates a series of exemplary figures relating to the extraction of pathophysiological pathway related features corresponding to calcifications within a blood vessel.

FIGS. 7A-7C illustrate figures of various embodiments relating to a performance of a disclosed heart failure assessment apparatus configured to utilize pathophysiological pathway related features to generate a medical prediction of heart failure risk for a patient.

FIG. 8 illustrates some additional embodiments of a heart failure assessment apparatus comprising a machine learning stage configured to utilize pathophysiological pathway related features and clinical information to generate a medical prediction.

FIG. 9 illustrates some additional embodiments of a heart failure assessment apparatus comprising a plurality of different machine learning stage configured to utilize pathophysiological pathway related features, clinical information, and/or demographic information to generate a medical prediction.

FIGS. 10-11 illustrate figures of various embodiments relating to a performance of a disclosed heart failure assessment apparatus configured to utilize pathophysiological pathway related features and demographic information to generate a medical prediction of heart failure risk for a patient.

FIG. 12 illustrates a flow diagram showing some additional embodiments of a method of utilizing pathophysiological pathway related features, clinical information, and/or demographic information to generate a medical prediction of heart failure risk for a patient.

FIG. 13 illustrates a block diagram of some embodiments of a heart failure assessment apparatus comprising a machine learning stage configured to utilize pathophysiological pathway related features to generate a medical prediction of heart failure risk for a patient.

### DETAILED DESCRIPTION

The description herein is made with reference to the drawings, wherein like reference numerals are generally utilized to refer to like elements throughout, and wherein the various structures are not necessarily drawn to scale. In the following description, for purposes of explanation, numerous specific details are set forth in order to facilitate understanding. It may be evident, however, to one of ordinary skill in the art, that one or more aspects described herein may be practiced with a lesser degree of these specific details. In other instances, known structures and devices are shown in block diagram form to facilitate understanding.

Heart failure occurs when a person's heart muscles don't pump blood as well as they should, causing the heart to fail to meet the body's needs. Heart failure can be caused by certain heart conditions that gradually leave a heart too weak and/or stiff to fill and pump blood properly. These conditions include narrowed arteries, high blood pressure, obesity, diabetes, and/or the like. Heart failure is a major cause of morbidity and mortality in the United States.

When the heart fails to pump properly, blood often backs up in other parts of a patient's body. For example, blood can collect in a patient's lungs, legs, and/or feet, resulting in swelling and/or other complications such as shortness of breath, heart palpitations, chest pain, etc. Patients that suffer from heart failure may receive medical treatment to improve the symptoms of heart failure. Since such medical treatment may help some patients to live longer, there is a compelling need for personalized pre-emptive heart failure risk prediction to facilitate precise preventive strategies. However, currently there are no widely validated models for heart failure risk prediction. The lack of validated models for heart failure risk prediction hinders pre-emptive initiation of therapies in at-risk patients.

The present disclosure relates an apparatus and/or method that utilizes a plurality of pathophysiological pathway related features, which have been extracted from digitized images (e.g., computed tomography calcium scoring (CTCS) images), to generate a medical prediction relating to heart failure risk. In some embodiments, the method may access digitized imaging data stored in a memory. A plurality of pathophysiological pathway related features are extracted from the digitized imaging data. The plurality of pathophysiological pathway related features correspond to one or more pathophysiological pathways relating to heart failure. The plurality of pathophysiological pathway related features are provided to a machine learning stage, which is configured to use the features to generate a medical prediction relating to heart failure risk. By utilizing the plurality of pathophysiological pathway related features to generate a medical prediction relating to heart failure risk, the disclosed heart failure assessment apparatus may provide health care professionals with a tool that can identify patients that are at a high risk of heart failure and that may benefit from preemptive treatment.

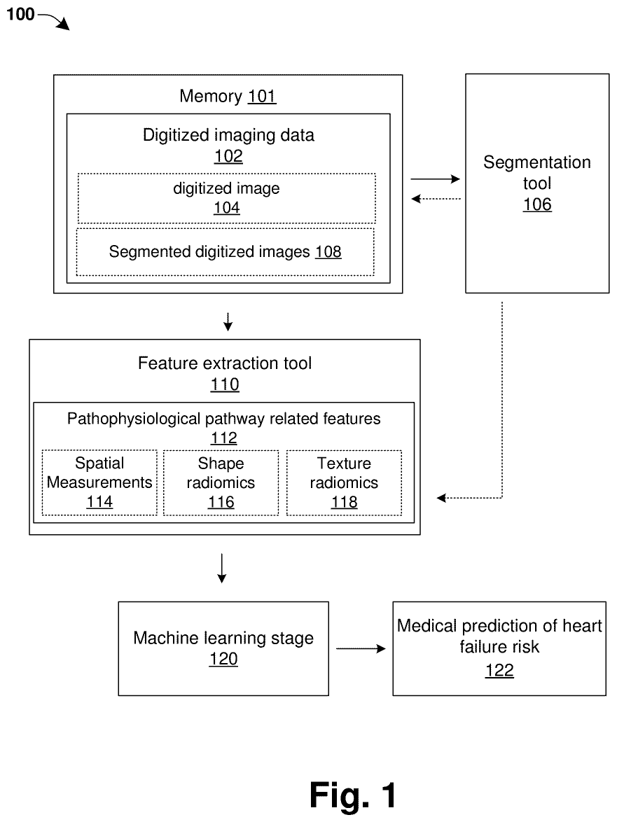

FIG. 1 illustrates some embodiments of a heart failure assessment apparatus 100 comprising a machine learning stage configured to utilize pathophysiological pathway related features to generate a medical prediction of heart failure risk.

The heart failure assessment apparatus 100 comprises a memory 101 configured to store digitized imaging data 102 for a patient that may be susceptible to heart failure. In some embodiments, the digitized imaging data 102 may comprise one or more non-contrast digitized images 104. The one or more non-contrast digitized images 104 may include cardiovascular computed tomography (CT) images, computed tomography calcium score (CTCS) images, and/or the like. For example, in various embodiments the one or more non-contrast digitized images 104 may comprise non-contrast, fast, low-dose computed tomography calcium score (CTCS) images, images of the chest (e.g., lung screening images), and/or the like.

In some embodiments, a segmentation tool 106 is in communication with the memory 101. The segmentation tool 106 is configured to segment the digitized imaging data 102 (e.g., the one or more non-contrast digitized images 104) to generate one or more segmented digitized images 108 that respectively identify one or more regions of interest (e.g., volumes of interest). In some embodiments, the one or more segmented digitized images 108 may be stored in the memory 101 as part of the digitized imaging data 102.

A feature extraction tool 110 is configured to extract a plurality of pathophysiological pathway related features 112 from the digitized imaging data 102 (e.g., from the one or more segmented digitized images 108). The plurality of pathophysiological pathway related features 112 are features that characterize pathways linked with heart failure pathogenesis. For example, the plurality of pathophysiological pathway related features 112 may include features that characterize the different regions of interest and/or changes (e.g., functional changes) in the different regions of interest that may be indicative of heart failure. In some embodiments, the plurality of pathophysiological pathway related features 112 may include spatial measurements 114, shape radiomic features 116, and texture radiomic features 118 extracted from the one or more regions of interest. In various embodiments, the plurality of pathophysiological pathway related features 112 may include first-order radiomic features (e.g., radiomic features generated based on single imaging unit values within a region of interest) and/or second-order radiomic features (e.g., radiomic features generated based on associations between neighboring imaging unit values within a region of interest) that have been selected to correspond to one or more pathophysiologic pathways for heart failure.

A machine learning stage 120 is configured to utilize the plurality of pathophysiological pathway related features 112 to generate a medical prediction of heart failure risk 122. It has been appreciated that by utilizing features that describe different pathophysiological pathways, the medical prediction of heart failure risk 122 may be more accurate than existing models. Therefore, the disclosed heart failure assessment apparatus 100 may provide health care professionals with a tool that can accurately identify patients that are at a high risk of heart failure and that may benefit from preemptive treatment (e.g., sodium-glucose co-transporter 2 inhibitors, non-steroidal mineralocorticoid antagonist Finerenone, etc.).

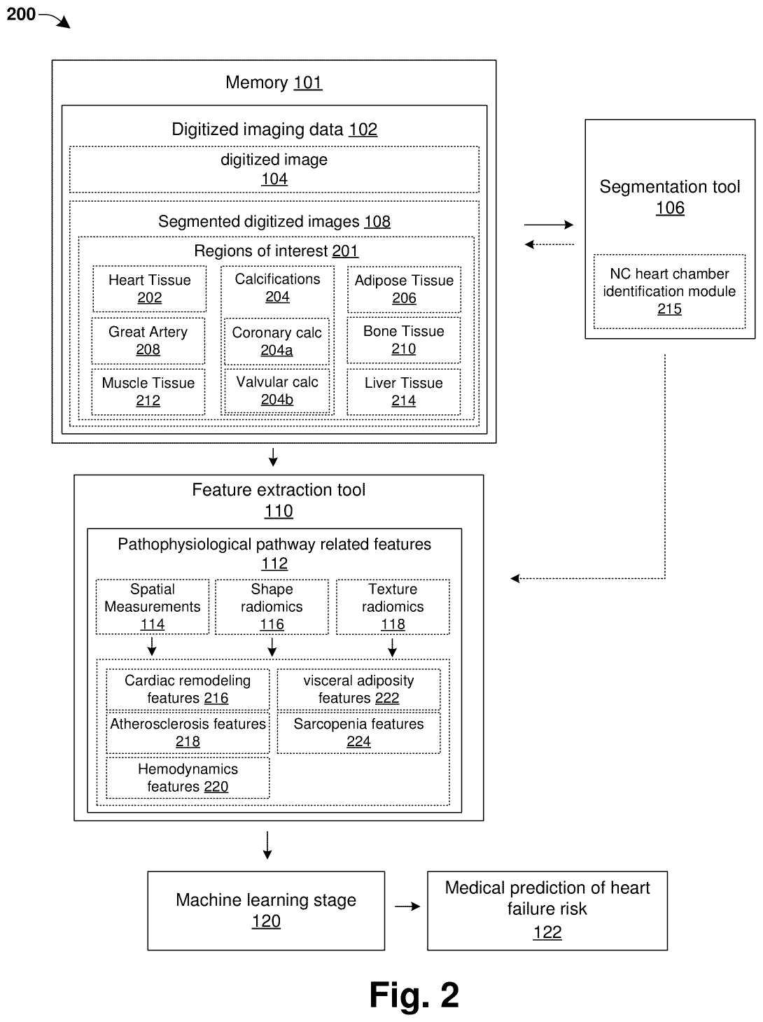

FIG. 2 illustrates some additional embodiments of a heart failure assessment apparatus 200 comprising a machine learning stage configured to utilize pathophysiological pathway related features to generate a medical prediction of heart failure risk.

The heart failure assessment apparatus 200 comprises a memory 101 configured to store digitized imaging data 102 for a patient that may be susceptible to heart failure. In some embodiments, the digitized imaging data 102 may comprise one or more non-contrast digitized images 104. The one or more non-contrast digitized images 104 may include cardiovascular computed tomography (CT) images. For example, in various embodiments the one or more non-contrast digitized images 104 may comprise non-contrast, fast, low-dose computed tomography calcium score (CTCS) images, images of the chest (e.g., lung screening images), and/or the like. In some embodiments, the one or more non-contrast digitized images 104 may be EKG (electrocardiogram) gated. In some embodiments, the memory 101 may comprise electronic memory (e.g., solid state memory, SRAM (static random-access memory), DRAM (dynamic random-access memory), and/or the like).

In some embodiments, a segmentation tool 106 is in communication with the memory 101. The segmentation tool 106 is configured to segment the one or more non-contrast digitized images 104 to generate one or more segmented digitized images 108 that respectively identify one or more regions of interest 201 (e.g., volumes of interest). In some embodiments, the one or more regions of interest 201 may comprise heart tissue 202 (e.g., a heart), calcifications 204 (e.g., coronary artery calcium, aortic valve calcifications, mitral valve calcification), adipose tissue 206 (e.g., epicardial adipose tissue, liver fat, visceral fat, subcutaneous fat, thoracic adipose depots, paracardial fat, periaortic fat, hepatic fat, and/or the like), great artery tissue 208 (e.g., aorta, branches of the aorta, pulmonary artery, branches of the pulmonary artery, etc.), bone tissue 210 (e.g., vertebrae, a vertebral body, etc.), muscle tissue 212 (e.g., spinal muscle, skeletal muscle, pectoralis muscle, etc.), liver tissue 214, and/or the like. In some embodiments, the calcifications 204 may comprise coronary calcifications 204a and valvular calcifications 204b.

In some embodiments, the one or more segmented digitized images 108 may comprise and/or be one or more binary masks. In some such embodiments, the one or more binary masks comprise images having a value of “1” in image units (e.g., pixels, voxels, etc.) identified as being within the one or more regions of interest 201 and having a value of “0” in image units outside of the one or more regions of interest 201.

In some embodiments, the segmentation tool 106 may comprise a deep learning model. For example, the segmentation tool 106 may comprise a graphical neural network (GNN) that utilizes an adaptive Hounsfield Unit (HU)-attention-window. In some embodiments, the segmentation tool 106 may utilize transformer models, which use a self-attention mechanism to differentially weigh a significance of each input data element. The self-attention mechanism may improve a GNN's ability to formulate geometric and semantic relationships between the regions of interest 201 (e.g., volumes of interest). In some embodiments, the deep learning model may be run on one or more processors (e.g., a central processing unit including one or more transistor devices configured to operate computer code to achieve a result, a microcontroller, or the like).

In some embodiments, the segmentation tool 106 may further comprise a non-contrast heart chamber identification module 215. Typically, heart chambers cannot be directly identified from non-contrast images. However, it has been appreciated that characteristics of the myocardium and/or four heart chambers (e.g., a ventricle size, shape, etc.) are prognostic of heart failure. The non-contrast heart chamber identification module 215 enables identification of additional regions of interest including the myocardium and/or the four heart chambers (i.e., the left atrium, right atrium, left ventricle, and the right ventricle) within a non-contrast digitized image. The identification of the myocardium and/or the four heart chambers as regions of interest can enhance a prognostic ability of the disclosed heart failure assessment apparatus 200. In some embodiments, the non-contrast heart chamber identification module 215 may comprise a deep learning segmentation model (e.g., an nnU-Net segmentation algorithm) that estimates shapes of the myocardium and/or the four heart chambers.

A feature extraction tool 110 is configured to extract a plurality of pathophysiological pathway related features 112 from the digitized imaging data 102 (e.g., from the one or more segmented digitized images 108). The plurality of pathophysiological pathway related features 112 may include features that have been selected to correspond to pathophysiologic pathways for heart failure. The plurality of pathophysiological pathway related features 112 may include spatial measurements 114, shape radiomic features 116, and/or texture radiomic features 118 taken from the one or more regions of interest 201. In some embodiments, the feature extraction tool 110 may be implemented as computer code run by a processing unit (e.g., a central processing unit including one or more transistor devices configured to operate computer code to achieve a result, a microcontroller, or the like).

The spatial measurements 114 may include morphological features (e.g., volume, surface area, shape), mass features (e.g., mass, center of mass, 2^{nd} moment) from segmented volumes of interest (e.g., calcifications, fat, muscle, etc.), depot-specific assessments (e.g., slice cross-sectional areas, thicknesses), hepatic attenuation and hepatic/spleen ratios (e.g., as estimates of hepatic fat content and/or EAT content percent surface area covered by a fat depot), and/or the like. The spatial measurements 114 may further include statistical measures (e.g., mean, median, standard deviation, maximum, minimum, kurtosis, large-bin histogram) taken over a plurality of regions of interest and/or segmented digitized images.

The shape radiomic features 116 and/or the texture radiomic features 118 may include first-order intensity features and texture features (e.g., Gabor filters with 4 scales and 4 orientations). In some embodiments, the shape radiomic features 116 may be extracted using cell clusters and/or cell graphs. In some embodiments, the texture radiomic features 118 may be extracted by measuring Hounsfield unit (HU) values. For example, the texture radiomic features 118 may be generated based upon HU values determined for exemplary fat depots (e.g., subcutaneous fat, epicardial fat, paracardial fat, visceral fat, and/or periaortic fat).

In various embodiments, the shape radiomic features 116 and/or the texture radiomic features 118 may include one or more of fat-omics (e.g., radiomic features extracted from segmented epicardial fat depots), bone-omics (e.g., radiomic features extracted from segmented bone), calcium omics (e.g., radiomic features extracted from segmented calcifications), and muscle-omics (e.g., radiomic features extracted from segmented muscle). Each radiomic feature may be associated with a specific aspect of heart failure. For example, calcium-omics may be associated with vascular pathophysiology, fat-omics may be associated with inflammation and localization, bone-omics may be associated with bone density (low-density associates with a high risk of heart failure) and/or frailty, etc.

In some embodiments, the plurality of pathophysiological pathway related features 112 (e.g., the spatial measurements 114, the shape radiomic features 116, and the texture radiomic features 118) may include one or more of cardiac remodeling features 216 (e.g., features relating to changes in a shape, size, mass, geometry, and/or function of a heart), atherosclerosis features 218 (e.g., features relating to coronary and/or vascular calcifications), hemodynamic features 220 (e.g., features relating to dynamic of blood flow, including an aorta and/or pulmonary artery size, a valvular calcification, etc.), visceral adiposity features 222 (e.g., features relating to liver fat and/or epicardial adipose fat), and sarcopenia features 224 (e.g., features relating to skeletal muscle and/or bone density). In other embodiments, the plurality of pathophysiological pathway related features 112 may include features related to other pathophysiological pathways (e.g., inflammation, strain, metabolic function, etc.).

A machine learning stage 120 is configured to utilize the plurality of pathophysiological pathway related features 112 to generate a medical prediction of heart failure risk 122. In some embodiments, the medical prediction of heart failure risk 122 may provide for an indication of both a risk of heart failure and/or a time to heart failure (e.g., a time interval from a CTCS scan to a first event of heart failure). In some embodiments, the machine learning stage 120 may comprise a regression model, a Cox Hazard regression model, a support vector machine (SVM), a linear discriminant analysis (LDA) classifier, a Naïve Bayes classifier, a Random Forest, Adaboost, or the like. In some embodiments, the machine learning stage 120 may be run on one or more processors (e.g., a central processing unit including one or more transistor devices configured to operate computer code to achieve a result, a microcontroller, or the like).

By using the plurality of pathophysiological pathway related features 112 to generate the medical prediction of heart failure risk 122, a risk of heart failure can be accurately assessed leading to improved treatment for patients. Furthermore, extracting the plurality of pathophysiological pathway related features 112 from non-contrast CTCS images offers a low-cost screening assessment for heart failure risk. For example, in some embodiments the disclosed heart failure assessment apparatus 200 can be used in conjunction with CTCS calcium screenings, so as to provide for an assessment of heart failure risk without additional patient imaging and/or significant cost. The low-cost screening assessment allows for the disclosed heart failure assessment apparatus 200 to be widely accessible for different medical treatment centers and patients. Furthermore, inclusion of the disclosed heart failure assessment with CTCS calcium screenings can provide for a periodic (e.g., annual) assessment of heart failure risk.

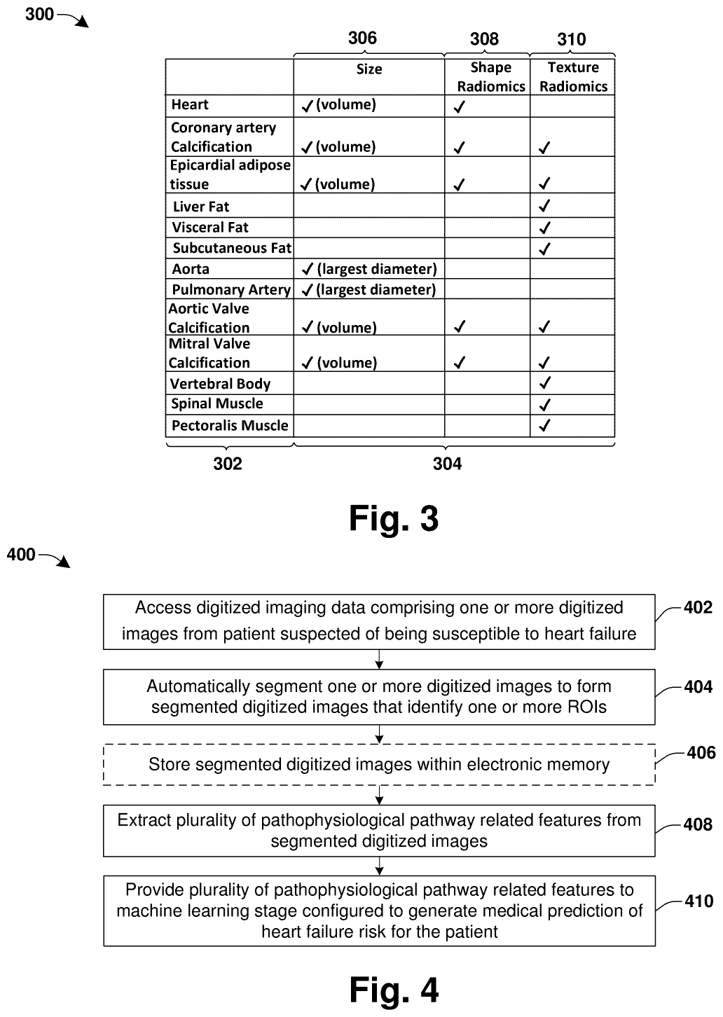

FIG. 3 illustrates a table 300 showing some exemplary pathophysiological pathway related features that may be utilized by a disclosed heart failure assessment apparatus to generate a medical prediction of heart failure risk. It will be appreciated that the pathophysiological pathway related features shown in table 300 are merely examples of pathophysiological pathway related features that may be used by a disclosed heart failure assessment system and are not limiting.

Table 300 includes features that convey specific facets of heart failure risk that consolidate imaging analogs of pathophysiologic pathways relevant to heart failure. Table 300 illustrates different regions of interest 302 and associated pathophysiological pathway related features 304. The different regions of interest 302 may include regions of interest that have been identified in a segmented digitized image (e.g., a segmented CTCS image). The pathophysiological pathway related features 304 associated with each region of interest include one or more of size features 306, shape radiomic features 308, and texture radiomic features 310. For example, pathophysiological pathway related features associated with a coronary artery calcification may include a volume of a coronary artery calcification, shape radiomic features extracted from the coronary artery calcification, and texture radiomic features extracted from the coronary artery calcification.

FIG. 4 illustrates a flow diagram showing some embodiments of a method 400 of utilizing pathophysiological pathway related features to generate a medical prediction of heart failure risk for a patient.

While the disclosed methods (e.g., method 400 and/or method 1200) are illustrated and described herein as a series of acts or events, it will be appreciated that the illustrated ordering of such acts or events are not to be interpreted in a limiting sense. For example, some acts may occur in different orders and/or concurrently with other acts or events apart from those illustrated and/or described herein. In addition, not all illustrated acts may be required to implement one or more aspects or embodiments of the description herein. Further, one or more of the acts depicted herein may be carried out in one or more separate acts and/or phases.

At act 402, digitized imaging data comprising one or more digitized images from a patient suspected of being susceptible to heart failure is accessed. In some embodiments, the digitized imaging data may comprise one or more non-contrast low-dose computed tomography calcium score (CTCS) images. In some embodiments, the patient may have high blood pressure, high cholesterol, obesity, diabetes, a high body mass index (BMI), and/or the like.

At act 404, the one or more digitized images are automatically segmented to form one or more segmented digitized images that identify one or more regions of interest (ROIs). In some embodiments, the one or more ROIs may comprise one or more of heart tissue, liver tissue, adipose tissue (e.g., thoracic adipose depots), great artery tissue, bone tissue (e.g., bone, vertebrae, etc.), muscle tissue, and/or calcifications (e.g., valvular calcifications, coronary calcifications, etc.). In some embodiments, the one or more digitized images may be segmented using a deep learning model.

At act 406, the one or more segmented digitized images may be stored within electronic memory, in some embodiments.

At act 408, a plurality of pathophysiological pathway related features are extracted from the one or more segmented digitized images. In some embodiments, the plurality of pathophysiological pathway related features comprise one or more of spatial measurements, shape radiomic features, and texture radiomic features extracted from the ROIs.

At act 410, the plurality of pathophysiological pathway related features are provided to a machine learning stage configured to generate a medical prediction of heart failure risk for the patient.

It will be appreciated that the disclosed methods and/or block diagrams may be implemented as computer-executable instructions, in some embodiments. Thus, in one example, a computer-readable storage device (e.g., a non-transitory computer-readable medium) may store computer executable instructions that if executed by a machine (e.g., computer, processor) cause the machine to perform the disclosed methods and/or block diagrams. While executable instructions associated with the disclosed methods and/or block diagrams are described as being stored on a computer-readable storage device, it is to be appreciated that executable instructions associated with other example disclosed methods and/or block diagrams described or claimed herein may also be stored on a computer-readable storage device.

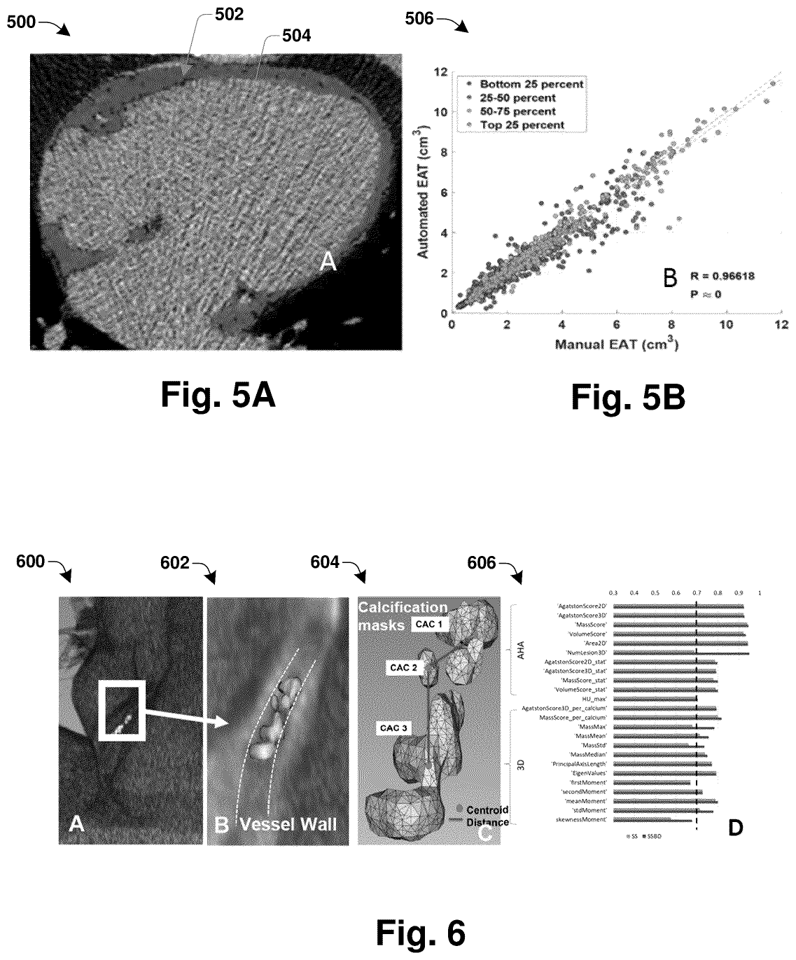

FIGS. 5A-5B illustrate figures of various embodiments showing automated segmentation of digitized images corresponding to this disclosure.

FIG. 5A illustrates some embodiments of a cross-sectional view 500 showing a segmented digitized image that identifies epicardial adipose tissue (EAT). The cross-sectional view 500 shows epicardial adipose tissue 502 as red and other tissue 504 as gray.

In some embodiments, a disclosed segmentation tool (e.g., segmentation tool 106 of FIG. 1) is configured to form a segmented digitized image using a look ahead slab-of-slices with bisection (“bisect”). For example, the segmentation tool may split a heart into halves. A lower half of the heart may be sequenced from bottom-to-middle and an upper half of the heart may be sequenced from top-to-middle. It has been appreciated that using a look ahead slab-of-slices with bisection is able to improve segmentation accuracy.

FIG. 5B shows some embodiments of a graph 506 illustrating a correlation between the disclosed automated segmentation of EAT and manual segmentation of EAT.

As shown in graph 506, a disclosed segmentation tool is able to achieve a very close correlation with manual segmentation of EAT (e.g., a dice score of 0.88 and an R-value of 0.98). Because the disclosed segmentation tool is able to achieve a very close correlation with manual segmentations of EAT, the disclosed segmentation tool can be used to accurately segment EAT, thereby allowing the disclosed heart failure assessment apparatus to generate a medical prediction of heart failure risk with minimal human intervention.

FIG. 6 illustrates a series of exemplary FIGS. 600-606 relating to the extraction of pathophysiological pathway related features corresponding to calcifications (e.g., calcium deposits) within a blood vessel.

Image 600 shows a digitized image of a blood vessel containing calcifications. Image 602 shows an annotated digitized image identifying a wall of the blood vessel that contains the calcifications. The disclosed method and apparatus are configured to extract features relating to the calcifications. In some embodiments, some extracted features relating to the calcifications (e.g., such as Agatston, mass, and volume scores) may be aggregated along a plurality of blood vessels (e.g., along each great artery).

As shown in image 604, in some embodiments calcification centroids may be used to calculate Euclidean distances between calcifications. For example, sub-voxel centroid locations may be used to calculate a calcified arterial distance as a sum of distances from a centroid to a centroid in consecutive sequential order.

Graph 606 shows 24 exemplary calcification features extracted from digitized images obtained from a plurality of patients. While graph 606 shows 24 exemplary calcification features, it will be appreciated that more calcification features (e.g., 90 or more calcification features) may be extracted from a digitized image. As shown in graph 606, the calcification features are divided into Accumulated Heart and Artery (AHA) features and 3D calcification features. The vertical dashed line shows a threshold above which the features are able to generate a prognosis having adequate reproducibility. In some embodiments, the use of blind deconvolution may improve intraclass correlation coefficient (ICC) in 21 out of 24 features. With blind deconvolution, 22 features are above threshold.

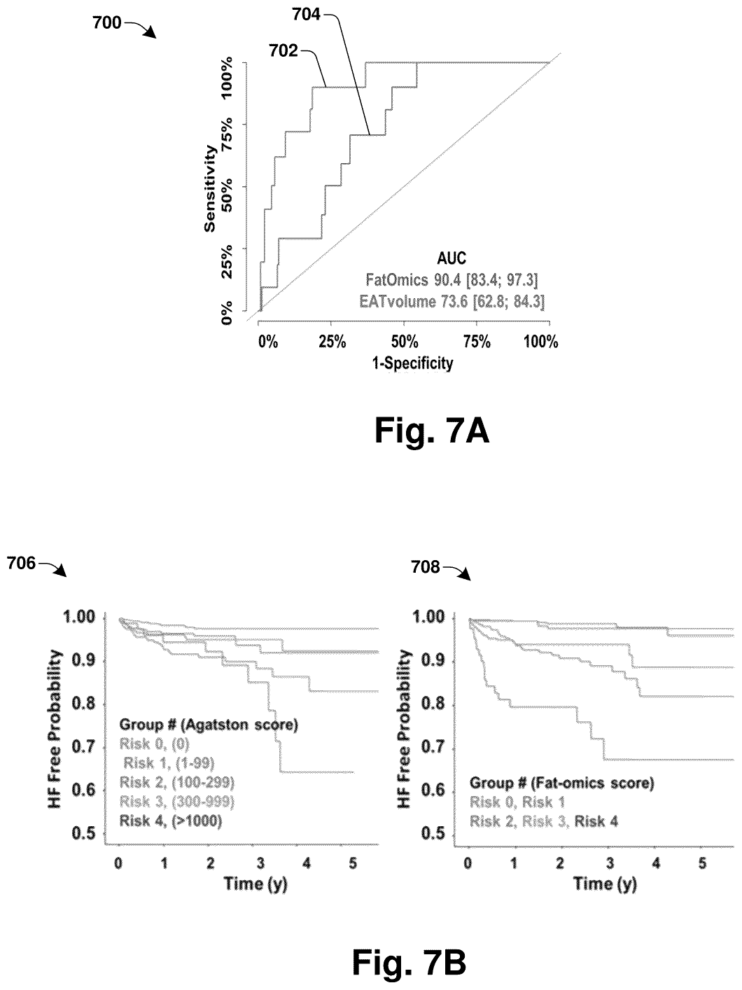

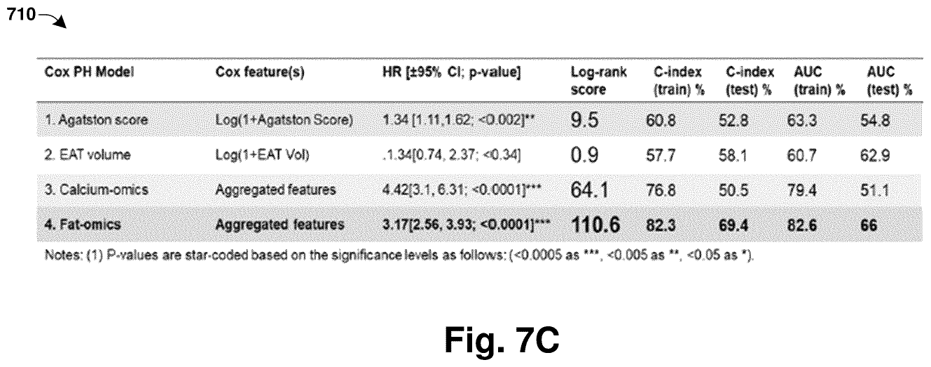

It has been appreciated that the pathophysiological pathway related features may have prognostic value for a disclosed machine learning stage when they describe either a single pathophysiological pathway or multiple pathophysiological pathways. For example, in some embodiments the plurality of pathophysiological pathway related features may be selected to correspond to one pathophysiologic pathway for heart failure (e.g., fat-omics). In other embodiments, the plurality of pathophysiological pathway related features may be selected to correspond to a plurality of pathophysiologic pathways for heart failure (e.g., cardiac remodeling features, atherosclerosis features, hemodynamic features, visceral adiposity features, and sarcopenia features). FIGS. 7A-7C illustrate exemplary performance metrics relating to a disclosed heart failure assessment apparatus configured to utilize pathophysiological pathway related features corresponding to different pathophysiological pathways for heart failure. The pathophysiological pathways illustrated in FIGS. 7A-7C are not limiting, but rather illustrate some examples of performance that may be achieved using different pathophysiological pathway related features corresponding to different pathophysiological pathways.

FIG. 7A shows some exemplary embodiments of a receiver operating characteristic (ROC) curve 700 illustrating performance of fat-omics and epicardial adipose tissue (EAT) volume.

As shown in the ROC curve 700, models based on fat-omics 702 (e.g., EAT radiomics) are superior to models based on EAT volume 704 for predicting heart failure, thereby indicating an importance of using fat-omics in a disclosed heart failure assessment system.

FIG. 7B shows exemplary graphs 706-708 illustrating performance of Agaston scores and fat-omics.

As shown in graph 706, Agaston is stratified according to recommended risk levels 0-4 with Agatston. Prior to year 4, the stratification is relatively small between the risk levels. As shown in graph 708, fat-omics is stratified into 0-4 risk levels having the same proportions of patients as Agatston. Comparison of graph 706 and graph 708 shows that models trained using fat-omics achieve better separation between risk levels. Therefore, fat-omics (e.g., EAT radiomics) provides superior discrimination of heart failure risk (e.g., especially between years 1 and 4) compared to the Agatston scores commonly used in clinical practice.

FIG. 7C illustrates a table 710 showing exemplary performance metrics associated with different types of pathophysiological pathway related features used to predict heart failure.

Table 710 illustrates performance metrics for Cox time-to-event models that perform risk prediction of heart failure based on epicardial fat-omics (e.g., radiomic features extracted from epicardial fat depots), calcium-omics (e.g., radiomic features extracted from calcifications), EAT (epicardial adipose tissue) volume, and Agatston score. As can be seen, Agatston provides for inferior performance in comparison to fat-omics, calcium-omics, and EAT volume. This further supports the use of pathophysiological pathway related features as a driver in predicting heart failure risk in a disclosed heart failure assessment apparatus.

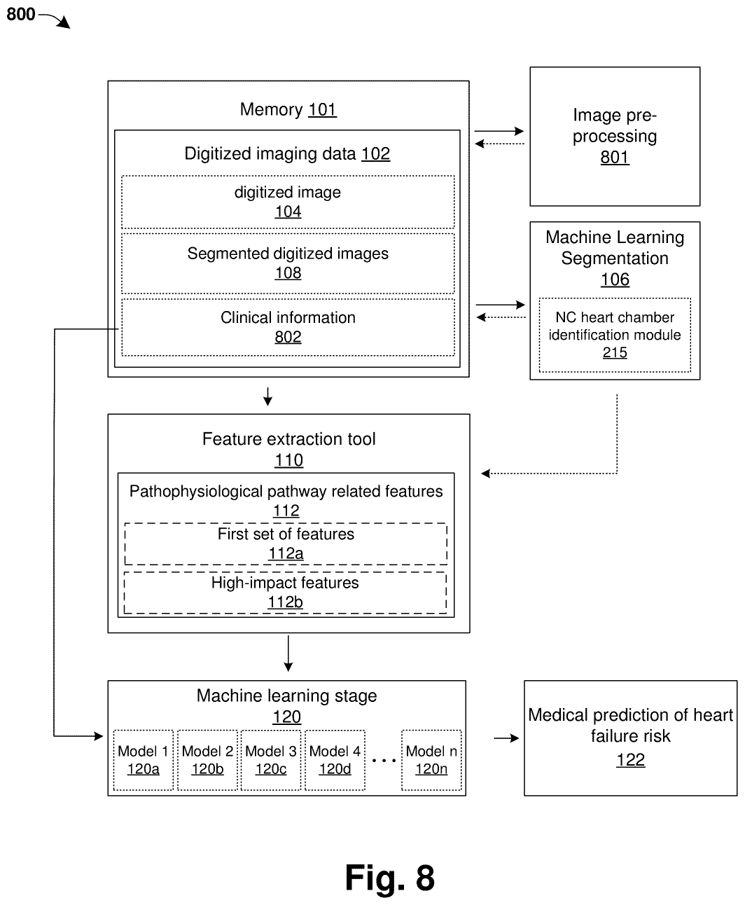

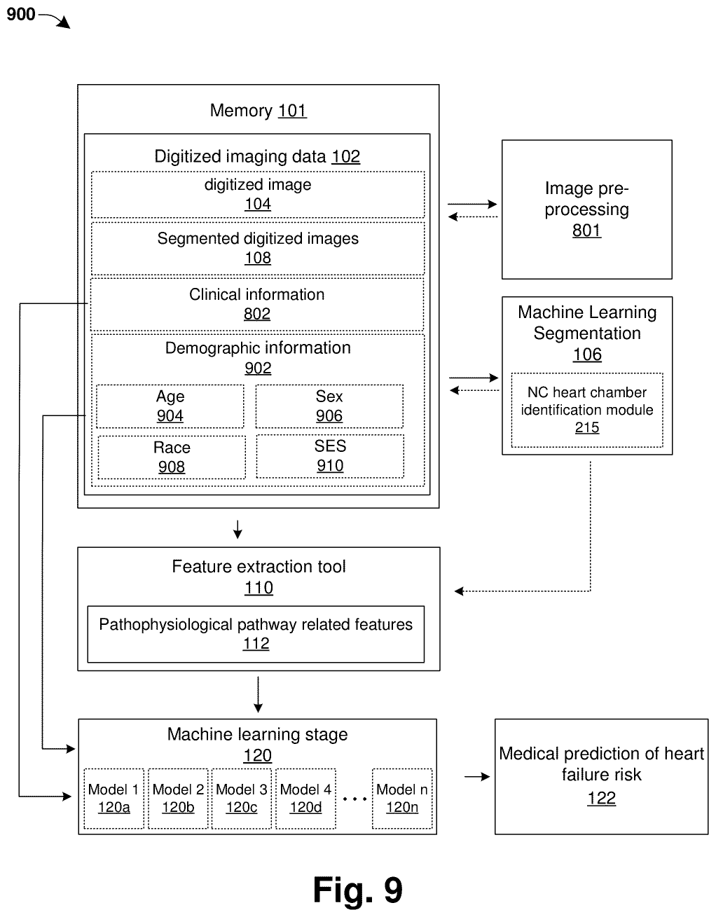

FIG. 8 illustrates some additional embodiments of a heart failure assessment apparatus 800 comprising a machine learning model configured to utilize pathophysiological pathway related features and clinical information to generate a medical prediction of heart failure risk.

The heart failure assessment apparatus 800 comprises a memory 101 configured to store digitized imaging data 102. The digitized imaging data 102 includes one or more non-contrast digitized images 104 (e.g., non-contrast CTCS images) for a patient that may be susceptible to heart failure. A segmentation tool 106 is configured to operate upon the digitized imaging data 102 to form a plurality of segmented digitized images 108. In some embodiments, the segmented digitized images 108 may be stored in the memory 101 as part of the digitized imaging data 102.

In some embodiments, an image pre-processing stage 801 may be configured to perform one or more pre-processing steps on the digitized imaging data 102 to improve image quality and/or subsequent feature extraction. In some embodiments, the image pre-processing stage 801 is configured to apply beam hardening correction to the digitized imaging data 102 to reduce beam hardening artifacts which can otherwise reduce Hounsfield unit (HU) values and interfere with accurate and precise feature extraction. In some embodiments, the image pre-processing stage 801 automatically determines correction parameters for a beam hardening correction model and applies them to reduce artifacts in an image. In other embodiments, the image pre-processing stage 801 may include deconvolution and/or the like.

A feature extraction tool 110 is configured to extract a plurality of pathophysiological pathway related features 112 from the one or more segmented digitized images 108. The plurality of pathophysiological pathway related features 112 may include features that have been selected to correspond to pathophysiologic pathways for heart failure. The plurality of pathophysiological pathway related features may include spatial measurements, shape radiomic features, and texture radiomic features taken from different regions of interest within the one or more segmented digitized images 108.

In some embodiments, during training the feature extraction tool 110 is configured to extract a first set of features 112a that include a first plurality of features. From the first set of features 112a, the feature extraction tool 110 may be configured to identify a plurality of high-impact features 112b that include a second plurality of features that is less than the first plurality of features. In some embodiments, the feature extraction tool 110 may identify the plurality of high-impact features 112b within the context of a time-to-event model (e.g., a Cox proportional hazard model). In some embodiments, the high-impact features may include a dispersion of calcifications, a number of calcifications, a calcification density, epicardial adipose tissue attenuation values closest to the myocardium, liver fat, etc.), and/or the like. After training is completed, the feature extraction tool 110 may extract the plurality of pathophysiological pathway related features 112 to be or to predominantly be the high-impact features 112b.

A machine learning stage 120 is configured to utilize the plurality of pathophysiological pathway related features 112 (e.g., the high-impact features 112b) to generate a medical prediction of heart failure risk 122.

In some embodiments, the memory 101 is further configured to store clinical information 802 relating to the patient. In various embodiments, the clinical information 802 may include one or more of systolic blood pressure, diastolic blood pressure, chronic kidney disease, smoking status, hypertension treatment, serum cholesterol, obesity, dyslipidemia, smoking status, cardiovascular risk factors, cardiovascular medications, body mass index (BMI), and the like. In some embodiments, the cardiovascular medications include one or more of statins, aspirin, betablockers, ACE inhibitors, blood pressure medications, heart rate medications, LDL-cholesterol, serum creatinine, and the like.

It has been appreciated that a patient's clinical information may affect the pathophysiological pathways that relate to heart failure for that patient. For example, a patient that is a smoker may have different pathophysiological pathways relating to heart failure than a non-smoker. The clinical information 802 stored in the memory 101 may therefore also affect the pathophysiological pathway related features 112 used by the machine learning stage 120 and/or a weighting of pathophysiological pathway related features that is performed by the machine learning stage 120. For example, certain pathophysiological pathway related features may be more prognostic for a person that has high blood pressure than for a person that has low blood pressure.

In some embodiments, a medical prediction of heart failure risk 122 for a patient may be generated by the machine learning stage 120 based upon both the plurality of pathophysiological pathway related features 112 (e.g., the high-impact features 112b) and clinical information relating to the patient. In some such embodiments, the machine learning stage 120 may comprise a plurality of different machine learning models 120a-120n that respectively correspond to different combinations of the clinical information 802, so as to account for differences in clinical information between patients. For example, the plurality of different machine learning models may comprise a first machine learning model 120a that has been trained to provide an accurate medical prediction of heart failure risk for a patient having high blood pressure, a second machine learning model 120b that has been trained to provide an accurate medical prediction of heart failure risk for a patient that is smoker, etc.

During operation, clinical information from a patient may be entered into the memory 101 and then used to subsequently determine which pathophysiological pathway related features 112 are provided to the machine learning stage 120 and which of the plurality of different machine learning models 120a-120n are used to generate the medical prediction of heart failure risk 122 for the patient. It has been appreciated that by utilizing the clinical information 802 to identify specific features and/or a specific machine learning model within the machine learning stage 120, the disclosed heart failure assessment apparatus 800 may provide for improved results.

FIG. 9 illustrates some additional embodiments of a heart failure assessment apparatus 900 comprising a machine learning model configured to utilize pathophysiological pathway related features, clinical information, and/or demographic information to generate a medical prediction of heart failure risk.

The heart failure assessment apparatus 900 comprises a memory 101 (e.g., electronic memory) configured to store digitized imaging data 102. The digitized imaging data 102 includes one or more non-contrast digitized images 104 (e.g., non-contrast CTCS images) for a patient that may be susceptible to heart failure. A segmentation tool 106 is configured to operate upon the digitized imaging data 102 to form a plurality of segmented digitized images 108. The segmented digitized images 108 may be stored in the memory 101.

The memory 101 is further configured to store clinical information 802 and demographic information 902 relating to the patient. In some embodiments, the demographic information 902 may include one or more of an age 904, a sex 906, a race 908, and a socioeconomic status (SES) 910. In some additional embodiments, the demographic information 902 may further include a geocode of residence, an insurance status, a diagnosis date, and/or the like.

A feature extraction tool 110 is configured to extract a plurality of pathophysiological pathway related features 112 from the digitized imaging data 102. The plurality of pathophysiological pathway related features 112 may include features that have been selected to correspond to pathophysiologic pathways for heart failure. The plurality of pathophysiological pathway related features 112 may include measurements, shape radiomic features, and texture radiomic features taken from different regions of interest within the one or more segmented digitized images 108.

A machine learning stage 120 is configured to utilize the plurality of pathophysiological pathway related features 112, the clinical information 802, and the demographic information 902 to generate a medical prediction relating to heart failure risk 122 for a patient. In some embodiments, the patient may belong to a demographic subgroup (e.g., an African American patient, an Asian American patient, etc.).

In some embodiments, the machine learning stage 120 may include a plurality of different machine learning models 120a-120n, which have been respectively trained to generate a medical prediction of heart failure risk for patients that have specific demographic information. For example, the machine learning stage 120 may comprise a first machine learning model 120a that has been trained to provide an accurate medical predication of heart failure risk for a first sex (e.g., that has been trained to be predictive for biological males), a second machine learning model 120b that has been trained to provide an accurate medical predication of heart failure risk for a second sex (e.g., that has been trained to be predictive for biological females), a third machine learning model 120c that has been trained to provide an accurate medical predication of heart failure risk for a first race (e.g., that has been trained to be predictive for Caucasians), a fourth machine learning model 120d that has been trained to provide an accurate medical predication of heart failure risk for a second race (e.g., that has been trained to be predictive for blacks), etc.

In some additional embodiments, the plurality of different machine learning models 120a-120n may also be trained to provide an accurate medical predication for different combinations of clinical information and demographic information. In some embodiments, one or more of the plurality of different machine learning models 120a-120n may comprise an a model that is generic to clinical and demographic information. This model may serve as a fallback option for combinations of clinical and/or demographic information that do not have specific models.

During operation, clinical and/or demographic information from a patient may be entered into the memory 101 and then used to subsequently determine which pathophysiological pathway related features 112 are provided to the machine learning stage 120 and which of the plurality of different machine learning models 120a-120n are used to generate the medical prediction relating to heart failure risk 122 for the patient. It has been appreciated that demographic information 902 (e.g., biological sex, race, and SES) plays a role in heart failure pathogenesis and risk prediction. The demographic information 902 stored in the memory 101 may therefore also affect the pathophysiological pathway related features 112 used by the machine learning stage 120 and/or a weighting of pathophysiological pathway related features that is performed by the machine learning stage 120. Thus, by utilizing the clinical information 802 and the demographic information 902 to identify specific features and/or a specific machine learning model within the machine learning stage 120, the disclosed heart failure assessment apparatus 900 may provide for improved results.

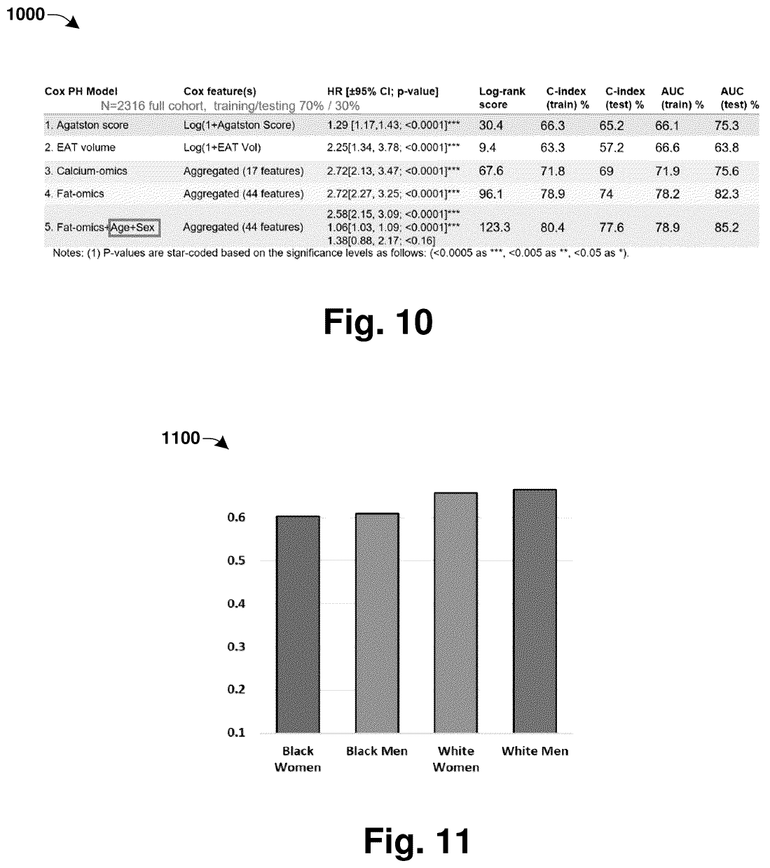

FIG. 10 illustrates an exemplary table 1000 relating to a performance of a disclosed heart failure assessment apparatus configured to utilize pathophysiological pathway related features and demographic information to generate a medical prediction of heart failure risk for a patient.

Table 1000 shows that both fat-omics and calcium-omics are able to achieve more predictive power for incident heart failure than an Agatston calcium score. The fat-omics result was obtained using an elastic net for feature reduction and Cox proportional hazard for time-to-event modeling with an internal 10-fold cross-validations. Furthermore, the use of fat-omics along with demographic information including sex and age in a prediction model increases a prediction accuracy.

FIG. 11 illustrates a bar graph 1100 showing exemplary areas under curve (AUCs) for patients having different races and sexes obtained by a disclosed heart failure assessment system using calcium radiomic features.

As can be seen in bar graph 1100, the area under the curve is generally lower for black patients than for white patients thereby indicating that a disclosed heart failure assessment system that does not specifically account for a patient's race will provide for results that may vary between races. Furthermore, the area under the curve is generally lower for female patients than for male patients thereby indicating that a disclosed heart failure assessment system that does not specifically account for a patient's sex will provide for results that may vary between sexes. By accounting for demographic information that includes race and sex, the disclosed heart failure assessment system can account for these biases and provide improved heart failure risk predictions for different demographic groups.

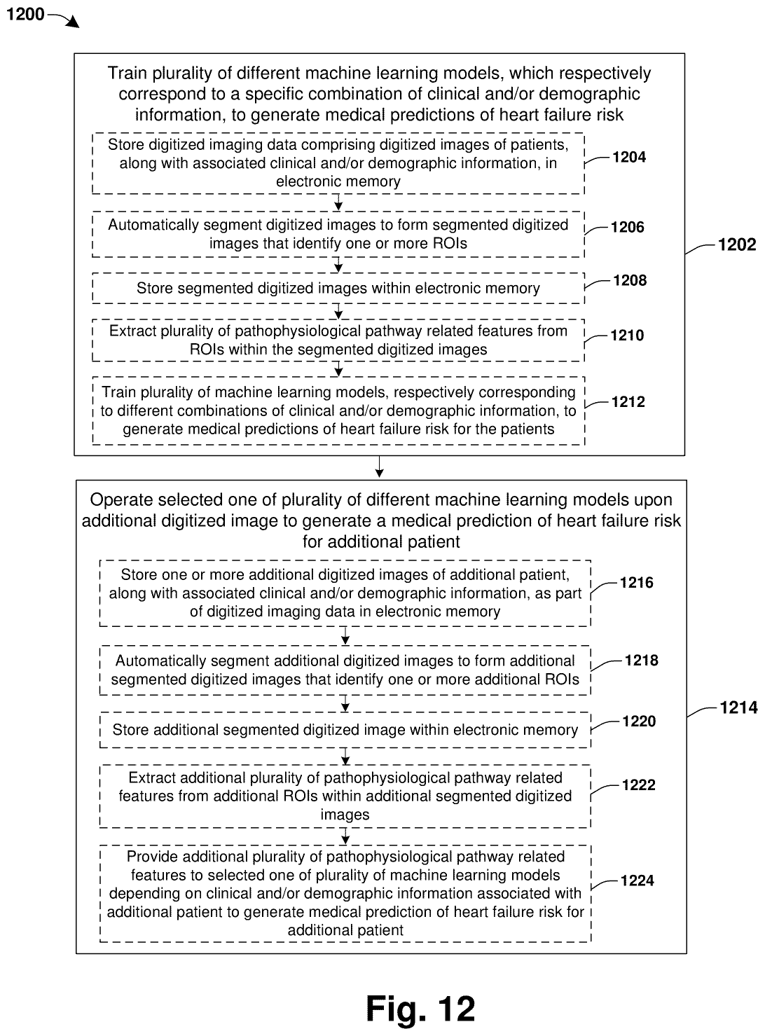

FIG. 12 illustrates a flow diagram showing some additional embodiments of a method 1200 of utilizing pathophysiological pathway related features, clinical information, and/or demographic information to generate a medical prediction of heart failure risk for a patient.

At act 1202, a plurality of different machine learning models, which respectively correspond to a specific combination of clinical and/or demographic information, are trained to generate a medical prediction of heart failure risk. In some embodiments, act 1202 may be performed according to acts 1204-1212.

At act 1204, digitized imaging data that comprises digitized images of a plurality of patients, and associated clinical and/or demographic information, is stored in electronic memory.

At act 1206, the digitized images are automatically segmented to form segmented digitized images that identify one or more regions of interest (ROIs). In some embodiments, the one or more ROIs may comprise one or more of heart tissue, liver tissue, adipose tissue (e.g., thoracic adipose depots), great artery tissue, bone tissue (e.g., bone, vertebrae, etc.), muscle tissue, and/or calcifications (e.g., valvular calcifications, coronary calcifications, etc.).

At act 1208, the segmented digitized images may be stored within the electronic memory as part of the digitized imaging data.

At act 1210, a plurality of pathophysiological pathway related features are extracted from the ROIs within the segmented digitized images. In some embodiments, the plurality of pathophysiological pathway related features comprise one or more of spatial measurements, shape radiomic features, and texture radiomic features extracted from the ROIs.

At act 1212, a plurality of machine learning models, which respectively correspond to different combinations of clinical and/or demographic information, are trained to generate medical predictions of heart failure risk for the patients. For example, the plurality of machine learning models may comprise models that are trained to provide accurate medical predictions for patients having specific races, genders, blood pressures, and/or the like.

At act 1214, an additional digitized image from an additional patient is operated upon by one of the plurality of different machine learning models, depending upon a specific combination of clinical and/or demographic information of the additional patient, to generate a medical prediction of heart failure risk for the additional patient. In some embodiments, act 1214 may be performed according to acts 1216-1224.

At act 1216, one or more additional digitized images of the additional patient, along with associated clinical and/or demographic information, are stored in the memory as part of the digitized imaging data.

At act 1218, the one or more additional digitized images are automatically segmented to form additional segmented digitized images that identify one or more additional regions of interest (ROIs). In some embodiments, the one or more additional ROIs may comprise one or more of heart tissue, liver tissue, adipose tissue (e.g., thoracic adipose depots), great arteries, bone tissue (e.g., bone, vertebrae, etc.), muscle tissue, and/or calcifications (e.g., valvular calcifications, coronary calcifications, etc.).

At act 1220, the additional segmented digitized images may be stored within the electronic memory as part of the digitized imaging data.

At act 1222, additional pathophysiological pathway related features are extracted from the additional ROIs within the additional segmented digitized images.

At act 1224, the additional plurality of pathophysiological pathway related features are provided to a selected one of the plurality of machine learning models depending on the clinical and/or demographic information associated with the additional patient. For example, if the additional patient is male, the additional plurality of pathophysiological related features may be provided to one of plurality of machine learning models that has been trained to generate an accurate medical prediction of a heart failure risk for a male patient. If the additional patient is female, the additional plurality of pathophysiological related features may be provided to a different one of plurality of machine learning models that has been trained to generate an accurate medical prediction of a heart failure risk for a female patient.

It will be appreciated that in some embodiments one of acts 1202 and 1214 may be excluded from the method 1200. For example, in some embodiments after a trained heart failure assessment apparatus has been developed, act 1202 may be skipped and the method may rather include act 1214.

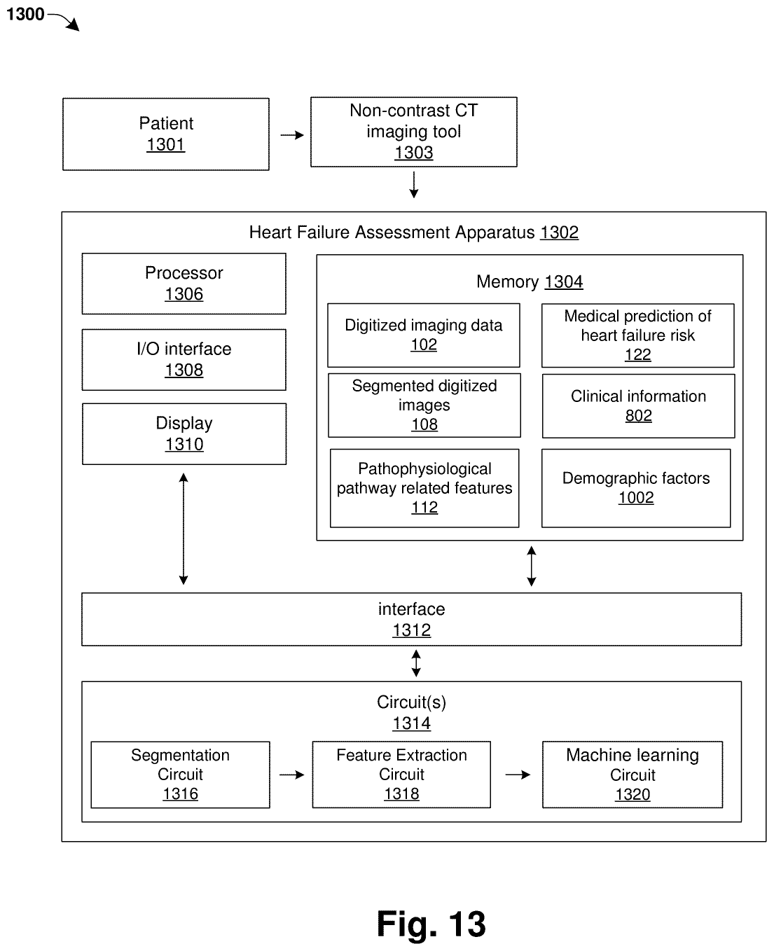

FIG. 13 illustrates a block diagram of some embodiments of a heart failure assessment apparatus 1300 comprising a machine learning stage configured to utilize pathophysiological pathway related features to generate a medical prediction of heart failure risk for a patient.

The heart failure assessment apparatus 1300 comprises a heart failure assessment apparatus 1302. The heart failure assessment apparatus 1302 is coupled to a non-contrast CT imaging tool 1303 that is configured to generate one or more non-contrast images (e.g., CTCS images) of a patient 1301. In some embodiments, the non-contrast CT imaging tool 1303 may comprise a low-dose CT scanner.

The heart failure assessment apparatus 1302 comprises a processor 1306 and a memory 1304. The processor 1306 can, in various embodiments, comprise circuitry such as, but not limited to, one or more single-core or multi-core processors. The processor 1306 can include any combination of general-purpose processors and dedicated processors (e.g., graphics processors, application processors, etc.). The processor(s) 1306 can be coupled with and/or can comprise memory (e.g., memory 1304) or storage and can be configured to execute instructions stored in the memory 1304 or storage to enable various apparatus, applications, or operating systems to perform operations and/or methods discussed herein.

The memory 1304 can be further configured to store digitized imaging data 102 comprising the one or more digitized images (e.g., non-contrast digitized images) obtained by the non-contrast CT imaging tool 1303. The one or more digitized images may comprise a plurality of pixels, each pixel having an associated intensity. In some additional embodiments, the one or more digitized images may be stored in the memory 1304 as one or more training sets of digitized images for training a classifier and/or one or more validation sets (e.g., test sets) of digitized images.

The heart failure assessment apparatus 1302 also comprises an input/output (I/O) interface 1308 (e.g., associated with one or more I/O devices), a display 1310, one or more circuits 1314, and an interface 1312 that connects the processor 1306, the memory 1304, the I/O interface 1308, the display 1310, and the one or more circuits 1314. The I/O interface 1308 can be configured to transfer data between the memory 1304, the processor 1306, the one or more circuits 1314, and external devices (e.g., non-contrast CT imaging tool 1303).

In some embodiments, the one or more circuits 1314 may comprise hardware components. In other embodiments, the one or more circuits 1314 may comprise software components. The one or more circuits 1314 can comprise a segmentation circuit 1316 (e.g., a deep learning circuit) configured to perform a segmentation operation on one or more digitized images within the digitized imaging data 102 to generate one or more segmented digitized images 108 respectively identifying one or more regions of interest (e.g., one or more of a heart, a liver, thoracic adipose depots, great arteries, skeletal and/or muscles, vertebrae, valvular calcifications, and coronary calcifications, and the like). In some embodiments, the one or more segmented digitized images 108 may comprise binary masks, which may be stored in the memory 1304.

In some additional embodiments, the one or more circuits 1314 may further comprise feature extraction circuit 1318 configured to extract a plurality of pathophysiological pathway related features 112 from the one or more segmented digitized images 108. The plurality of pathophysiological pathway related features 112 may be stored in the memory 1304.

In some embodiments, the one or more circuits 1314 may further comprise a machine learning circuit 1320 configured to operate one or more machine learning models (e.g., a Cox-proportional Hazard model) upon the plurality of pathophysiological pathway related features 112 to generate a medical prediction of heart failure risk 122.

In some embodiments, the machine learning circuit 1320 may be configured to selectively operate a plurality of different machine learning models upon different sets pathophysiological pathway related features. In such embodiments, the plurality of different machine learning models respectively correspond to a specific combination of clinical information 802 and/or demographic information 1002 stored in the memory 1304. During operation, the machine learning circuit 1320 may operate one of the plurality of different machine learning models upon different pathophysiological pathway related features extracted from one or more images of a patient, based upon the clinical information and/or demographic information relating to the patient, to generate the medical prediction of heart failure risk 122 for the patient.

Therefore, the present disclosure relates to a method and associated apparatus that utilizes a plurality of pathophysiological pathway related features, which have been extracted from digitized imaging data (e.g., low-dose computed tomography (CT) calcium score (CTCS) images), to generate a medical prediction of heart failure risk.

In some embodiments, the present disclosure relates to a method, the method includes accessing digitized imaging data stored in a memory, the digitized imaging data corresponding to a patient; extracting a plurality of pathophysiological pathway related features from the digitized imaging data, the plurality of pathophysiological pathway related features corresponding to one or more pathophysiological pathways relating to heart failure; and providing the plurality of pathophysiological pathway related features to a machine learning stage, the machine learning stage being configured to generate a medical prediction of heart failure risk for the patient using the plurality of pathophysiological pathway related features.

In other embodiments, the present disclosure relates to an apparatus, the apparatus includes a memory configured to store digitized imaging data of a patient, the digitized imaging data including one or more segmented digitized images that identify one or more of adipose tissue, bone tissue, muscle tissue, calcifications, great artery tissue, heart tissue, and liver tissue; a feature extraction tool configured to extract a plurality of pathophysiological pathway related features from the digitized imaging data, the plurality of pathophysiological pathway related features including spatial measurements, shape radiomic features, and texture radiomic features; and a machine learning stage configured to generate a medical prediction of heart failure for the patient based upon the plurality of pathophysiological pathway related features.

In yet other embodiments, the present disclosure relates to a non-transitory computer-readable medium storing computer-executable instructions that, when executed, cause a processor to perform operations, including accessing digitized imaging data stored in a memory, the digitized imaging data including a digitized image corresponding to a patient; extracting a plurality of pathophysiological pathway related features from the digitized imaging data, the plurality of pathophysiological pathway related features including spatial measurements, shape radiomic features, and texture radiomic features corresponding to one or more of heart tissue, adipose tissue, bone tissue, muscle tissue, calcifications, great artery tissue, and liver tissue; and providing the plurality of pathophysiological pathway related features to a machine learning stage that has been trained to generate a medical prediction of heart failure risk for the patient

Examples herein can include subject matter such as an apparatus, including a digital whole slide scanner, a CT system, an MRI system, a personalized medicine system, a CADx system, a processor, a system, circuitry, a method, means for performing acts, steps, or blocks of the method, at least one machine-readable medium including executable instructions that, when performed by a machine (e.g., a processor with memory, an application-specific integrated circuit (ASIC), a field programmable gate array (FPGA), or the like) cause the machine to perform acts of the method or of an apparatus or system, according to embodiments and examples described.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

“Computer-readable storage device”, as used herein, refers to a device that stores instructions or data. “Computer-readable storage device” does not refer to propagated signals. A computer-readable storage device may take forms, including, but not limited to, non-volatile media, and volatile media. Non-volatile media may include, for example, optical disks, magnetic disks, tapes, and other media. Volatile media may include, for example, semiconductor memories, dynamic memory, and other media. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a memory stick, and other media from which a computer, a processor or other electronic device can read.

“Circuit”, as used herein, includes but is not limited to hardware, firmware, software in execution on a machine, or combinations of each to perform a function(s) or an action(s), or to cause a function or action from another logic, method, or system. A circuit may include a software controlled microprocessor, a discrete logic (e.g., ASIC), an analog circuit, a digital circuit, a programmed logic device, a memory device containing instructions, and other physical devices. A circuit may include one or more gates, combinations of gates, or other circuit components. Where multiple logical circuits are described, it may be possible to incorporate the multiple logical circuits into one physical circuit. Similarly, where a single logical circuit is described, it may be possible to distribute that single logical circuit between multiple physical circuits.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A method, comprising:
accessing digitized imaging data stored in a memory, the digitized imaging data corresponding to a patient;
extracting a plurality of pathophysiological pathway related features from the digitized imaging data, wherein the plurality of pathophysiological pathway related features correspond to one or more pathophysiological pathways relating to heart failure; and
providing the plurality of pathophysiological pathway related features to a machine learning stage, wherein the machine learning stage is configured to generate a medical prediction of heart failure risk for the patient using the plurality of pathophysiological pathway related features.

2. The method of claim 1, wherein the plurality of pathophysiological pathway related features are extracted from one or more regions of interest including one or more of heart tissue, liver tissue, adipose tissue, great artery tissue, calcifications, bone tissue, and muscle tissue.

3. The method of claim 2, wherein the calcifications include coronary calcifications and valvular calcifications.

4. The method of claim 1, wherein the plurality of pathophysiological pathway related features include spatial measurements, shape radiomic features, and texture radiomic features.

5. The method of claim 1, wherein the plurality of pathophysiological pathway related features include one or more of cardiac remodeling features, atherosclerosis features, hemodynamic features, visceral adiposity features, and sarcopenia features.

6. The method of claim 1, further comprising:
providing the plurality of pathophysiological pathway related features to a specific machine learning model of a plurality of machine learning models within the machine learning stage, based upon clinical information relating to the patient, wherein the plurality of machine learning models respectively have been trained to provide accurate results for a specific combination of clinical information.

7. The method of claim 6, wherein the clinical information includes one or more of obesity, diabetes, hypertension, dyslipidemia, chronic kidney disease, cardiovascular medications, and smoking status of the patient.

8. The method of claim 7, wherein the cardiovascular medications include one or more of statins, aspirin, betablockers, ACE inhibitors, blood pressure medications, heart rate medications, LDL-cholesterol, serum creatinine.

9. The method of claim 1, further comprising:
providing the plurality of pathophysiological pathway related features to a specific machine learning model of a plurality of machine learning models within the machine learning stage, based upon demographic information relating to the patient, wherein the plurality of machine learning models respectively have been trained to provide accurate results for a specific combination of demographic information.

10. The method of claim 9, wherein the demographic information includes one or more of an age, a race, a sex, a geocode of residence, an insurance status, a diagnosis date, and a socioeconomic status of the patient.

11. The method of claim 1, wherein the digitized imaging data includes a computed tomography calcium scoring (CTCS) image.

12. The method of claim 1, further comprising:
segmenting the digitized imaging data to form segmented digitized images that identify one or more of heart tissue, great artery tissue, adipose tissue, liver tissue, bone tissue, muscle tissue, and calcifications; and
storing the segmented digitized images in the memory as part of the digitized imaging data.

13. The method of claim 12, wherein the digitized imaging data is segmented using a deep learning model including a graphical neural network (GNN).

14. A heart failure assessment system, comprising:
a memory configured to store digitized imaging data of a patient, wherein the digitized imaging data includes one or more segmented digitized images that identify one or more of adipose tissue, bone tissue, muscle tissue, calcifications, great artery tissue, heart tissue, and liver tissue;
a feature extraction tool configured to extract a plurality of pathophysiological pathway related features from the digitized imaging data, wherein the plurality of pathophysiological pathway related features comprise spatial measurements, shape radiomic features, and texture radiomic features; and
a machine learning stage configured to generate a medical prediction of heart failure for the patient based upon the plurality of pathophysiological pathway related features.

15. The heart failure assessment system of claim 14,
wherein the memory is further configured to store demographic information relating to the patient; and
wherein the machine learning stage comprises a plurality of machine learning models, the plurality of machine learning models respectively trained to generate the medical prediction of heart failure for a specific set of demographic information; and
wherein the plurality of pathophysiological pathway related features are provided to one of the plurality of machine learning models depending upon the demographic information.

16. The heart failure assessment system of claim 14, wherein the medical prediction of heart failure includes a time to heart failure.

17. The heart failure assessment system of claim 14,
wherein the memory is further configured to store clinical information relating to the patient; and
wherein the machine learning stage comprises a plurality of machine learning models, the plurality of machine learning models respectively trained to generate the medical prediction relating to heart failure for a specific set of clinical information; and
wherein the plurality of pathophysiological pathway related features are provided to one of the plurality of machine learning models depending upon the clinical information.

18. The heart failure assessment system of claim 17, wherein the clinical information includes one or more of systolic blood pressure, diastolic blood pressure, chronic kidney disease, smoking status, hypertension treatment, serum cholesterol, cardiovascular risk factors, cardiovascular medications, and body mass index.

19. A non-transitory computer-readable medium storing computer-executable instructions that, when executed, cause a processor to perform operations, comprising:
accessing digitized imaging data stored in a memory, wherein the digitized imaging data comprises a digitized image corresponding to a patient;
extracting a plurality of pathophysiological pathway related features from the digitized imaging data, wherein the plurality of pathophysiological pathway related features comprise spatial measurements, shape radiomic features, and texture radiomic features corresponding to one or more of heart tissue, adipose tissue, bone tissue, muscle tissue, calcifications, great artery tissue, and liver tissue; and
providing the plurality of pathophysiological pathway related features to a machine learning stage that has been trained to generate a medical prediction of heart failure risk for the patient.

20. The non-transitory computer-readable medium of claim 19, wherein the operations further comprise:
storing demographic information relating to the patient in the memory; and
providing the plurality of pathophysiological pathway related features to a specific machine learning model within the machine learning stage, based upon the demographic information relating to the patient.
